(19) United States
(12) Patent Application Publication
(10) Pub. No.: US 2024/0091439 A1
(43) Pub. Date: Mar. 21, 2024

(54) SYSTEM FOR REGULATING PRESSURE IN AN INTRAVENOUS FLUID SUPPLY

(71) Applicant: HONEYWELL INTERNATIONAL INC., Charlotte, NC (US)

(72) Inventors: HariPrasad PM, Charlotte, NC (US); Manjunatha HM, Charlotte, NC (US); Nikhil PS, Charlotte, NC (US)

(21) Appl. No.: 18/460,007

(22) Filed: Sep. 1, 2023

(30) Foreign Application Priority Data
Sep. 16, 2022 (IN) 202211053089

(51) Int. Cl. A61M 5/172; A61M 5/142

(52) U.S. Cl. CPC A61M 5/172; A61M 5/14212; A61M 2205/3331

(57) ABSTRACT

A system for regulating the pressure of an intravenous fluid supply is provided. An example intravenous fluid supply system may include an intravenous fluid supply tube supplying intravenous fluid to a patient from an intravenous fluid supply bag. A pressure may be applied to the intravenous fluid supply bag by a pressure bag adjacent to the intravenous fluid supply bag. A pump may be fluidly connected to the pressure bag, such that a fluid may be pumped into the pressure bag, applying pressure to the intravenous fluid supply bag. The system may further include a pressure sensor positioned to measure a pressure within the intravenous fluid supply tube. The intravenous fluid supply may include a controller communicatively connected to the pressure sensor and the pump, wherein the controller regulates an output of the pump based at least in part on one or more measurements of the pressure sensor.

### CROSS-REFERENCE TO RELATED APPLICATIONS

This application claims priority pursuant to 35 U.S.C. 119(a) to Indian Application No. 202211053089, filed Sep. 16, 2022, which application is incorporated herein by reference in its entirety.

### Technological Field

Embodiments of the present disclosure relate generally to intravenous (IV) fluid supply systems, and more particularly, to regulating the output fluid pressure of an IV fluid supply.

### BACKGROUND

Applicant has identified many technical challenges and difficulties associated with maintaining a consistent output pressure when administering an IV fluid to a patient. Through applied effort, ingenuity, and innovation, Applicant has solved problems related to regulating the pressure of an IV fluid supply by developing solutions embodied in the present disclosure, which are described in detail below.

### BRIEF SUMMARY

Various embodiments are directed to an example system, method, and computer program product for regulating the pressure of an intravenous fluid supply in an intravenous fluid supply system.

In accordance with some embodiments of the present disclosure, an example intravenous fluid supply system is provided. In some embodiments, the intravenous fluid supply system may comprise an intravenous fluid supply tube in fluid connection with an outlet port of an intravenous fluid supply bag containing an intravenous fluid. The intravenous fluid may be ejected into the intravenous fluid supply tube upon the application of a pressure on the intravenous fluid supply bag. In some embodiments, the intravenous fluid supply system may further comprise a pump fluidly connected to an inlet port of a pressure bag, wherein in an instance in which the pressure bag is adjacent to the intravenous fluid supply bag, and in an instance in which a fluid is pumped into the inlet port of the pressure bag by the pump, pressure may be applied to the adjacent intravenous fluid supply bag. In some embodiments, the intravenous fluid supply system may further comprise a pressure sensor positioned to measure a pressure within the intravenous fluid supply tube. Further, in some embodiments, the intravenous fluid supply system may comprise a controller communicatively connected to the pressure sensor and the pump, wherein the controller regulates an output of the pump based at least in part on one or more measurements of the pressure sensor.

In some embodiments, the intravenous fluid supply tube may be fluidly connected to a blood vessel of a patient, wherein the pressure sensor measures the pressure of the intravenous fluid in the intravenous fluid supply tube between the intravenous fluid supply bag and the blood vessel of the patient.

In some embodiments, the controller may regulate the output of the pump such that the pressure of the intravenous fluid in the intravenous fluid supply tube may be greater than a blood pressure of the patient.

In some embodiments, a cannula may be inserted into the blood vessel of the patient, and the pressure sensor may measure the pressure in the intravenous fluid supply tube between the patient and the cannula.

In some embodiments, the pressure sensor may measure a pressure measurement of the intravenous fluid in the intravenous fluid supply tube and transmit the pressure measurement to the controller.

In some embodiments, in an instance in which the pressure measurement drops below a minimum pressure threshold, the controller may adjust the output of the pump.

In some embodiments, the minimum pressure threshold may be between 275 and 285 millimeters of mercury and the pressure of the intravenous fluid in the intravenous fluid supply tube may be maintained between 275 millimeters of mercury and 310 millimeters of mercury.

In some embodiments, regulating the output of the pump may comprise activating and deactivating the pump.

In some embodiments, the controller may transmit the one or more measurements of the pressure sensor to a display device.

In some embodiments, intravenous fluid supply system may further comprise an additional pressure sensor, wherein the additional pressure sensor is fluidly connected to the pressure bag through a second outlet port, wherein the additional pressure sensor is communicatively connected to the controller, and wherein the controller regulates the output of the pump based at least in part on one or more measurements of the additional pressure sensor.

An example method for regulating a pressure of an intravenous fluid supply is further provided. In some embodiments, the method may comprise receiving a pressure measurement of a plurality of pressure measurements from a pressure sensor, wherein the pressure sensor is positioned to measure the pressure within an intravenous fluid supply tube, and wherein the intravenous fluid supply tube is fluidly connected to an outlet port of an intravenous fluid supply bag. The method may further comprise determining an output of a pump, fluidly connected to an inlet port of a pressure bag, based at least in part on the pressure measurement received from the pressure sensor, wherein the pressure bag is adjacent to an intravenous fluid supply bag containing an intravenous fluid, and wherein in an instance in which a fluid is pumped into the inlet port of the pressure bag, pressure is applied to the intravenous fluid supply bag, such that, intravenous fluid is ejected from an outlet port into the intravenous fluid supply tube. The method may further comprise altering the output of the pump based at least in part on the pressure measurement received from the pressure sensor.

In some embodiments, the intravenous fluid supply tube may be fluidly connected to a blood vessel of a patient and the pressure sensor may measure the pressure of the intravenous fluid in the intravenous fluid supply tube between the intravenous fluid supply bag and the blood vessel of the patient.

In some embodiments, the controller may regulate the output of the pump such that the pressure of the intravenous fluid in the intravenous fluid supply tube is greater than a blood pressure of the patient.

In some embodiments, determining the output of the pump may further comprise comparing the pressure measurement received from the pressure sensor to a minimum pressure threshold, and reconfiguring the output of the pump if the measurement received from the pressure sensor is below the minimum pressure threshold.

In some embodiments, altering the output of the pump may comprise activating and deactivating the pump.

In some embodiments, the method may further comprise transmitting the plurality of pressure measurements of the pressure sensor for display on a display device.

An example computer program product for regulating a pressure of an intravenous fluid supply is further provided. In some embodiments, the computer program product may comprise at least one non-transitory computer-readable storage medium having computer-readable program code portions stored therein, the computer-readable program code portions comprising an executable portion configured to receive a pressure measurement of a plurality of pressure measurements from a pressure sensor, wherein the pressure sensor is positioned to measure the pressure within an intravenous fluid supply tube, and wherein the intravenous fluid supply tube is fluidly connected to an outlet port of an intravenous fluid supply bag. In some embodiments, the executable portion may be further configured to determine an output of a pump, fluidly connected to an inlet port of a pressure bag, based at least in part on the pressure measurement received from the pressure sensor, wherein the pressure bag is adjacent to an intravenous fluid supply bag containing an intravenous fluid, and wherein in an instance in which a fluid is pumped into the inlet port of the pressure bag, pressure is applied to the intravenous fluid supply bag, such that, intravenous fluid is ejected from an outlet port into the intravenous fluid supply tube.

In some embodiments, the executable portion may be further be configured to alter the output of the pump based at least in part on the pressure measurement received from the pressure sensor.

In some embodiments, the intravenous fluid supply tube may be fluidly connected to a blood vessel of a patient, wherein the pressure sensor measures the pressure of the intravenous fluid in the intravenous fluid supply tube between the intravenous fluid supply bag and the blood vessel of the patient.

In some embodiments, determining the output of the pump may further comprise comparing the pressure measurement received from the pressure sensor to a minimum pressure threshold, and reconfiguring the output of the pump if the measurement received from the pressure sensor is below the minimum pressure threshold.

In some embodiments, altering the output of the pump may comprise activating and deactivating the pump.

### BRIEF DESCRIPTION OF THE DRAWINGS

Reference will now be made to the accompanying drawings. The components illustrated in the figures may or may not be present in certain embodiments described herein. Some embodiments may include fewer (or more) components than those shown in the figures in accordance with an example embodiment of the present disclosure.

FIG. 1A illustrates an overall view of an example IV fluid supply system in accordance with an example embodiment of the present disclosure.

FIG. 1B illustrates another example IV fluid supply system in accordance with an example embodiment of the present disclosure.

FIG. 1C illustrates a close-up view of an example IV fluid supply bag and connected components in accordance with an example embodiment of the present disclosure.

FIG. 2 illustrates a cross-sectional view of an example pressure sensor of an IV fluid supply system in accordance with an example embodiment of the present disclosure.

FIG. 3A illustrates an example pump and connected components of an IV fluid supply system in accordance with an example embodiment of the present disclosure.

FIG. 3B illustrates another example pump and connected components, including an additional pressure sensor, of an IV fluid supply system in accordance with an example embodiment of the present disclosure.

FIG. 4A illustrates an example IV fluid supply system transmitting IV fluid to a patient in accordance with an example embodiment of the present disclosure.

FIG. 4B illustrates another example IV fluid supply system, including an additional pressure sensor, transmitting IV fluid to a patient in accordance with an example embodiment of the present disclosure.

FIG. 5 illustrates an example block diagram showing example components of a controller in accordance with an example embodiment of the present disclosure.

FIG. 6 depicts a flowchart illustrating an example control loop for regulating the IV fluid supply pressure in an IV fluid supply system in accordance with an example embodiment of the present disclosure.

FIG. 7 depicts a flowchart illustrating an example operation performed by a controller in regulating the IV fluid supply pressure in an IV fluid supply system in accordance with an example embodiment of the present disclosure.

### DETAILED DESCRIPTION

Example embodiments will be described more fully hereinafter with reference to the accompanying drawings, in which some, but not all embodiments of the inventions of the disclosure are shown. Indeed, embodiments of the disclosure may be embodied in many different forms and should not be construed as limited to the embodiments set forth herein; rather, these embodiments are provided so that this disclosure will satisfy applicable legal requirements Like numbers refer to like elements throughout.

Various example embodiments address technical problems associated with regulating the fluid pressure when providing an IV fluid supply to a patient. As understood by those of skill in the field to which the present disclosure pertains, there are numerous example scenarios in which a user may need to provide an IV fluid supply to a patient at a regulated pressure.

In general, an IV fluid supply system may be utilized to provide fluid directly into the blood vessels of a receiving patient. A medical professional may use a cannula containing a needle to inject a small tube, or catheter directly into a patient's blood vessel. The catheter may be fluidly connected to an IV fluid supply, for example, an IV fluid supply bag. The IV fluid supply facilitates the administration of the IV fluid directly into the patient's blood vessel. The IV fluid may contain vitamins, minerals, nutrients, medications, and or other substances needed by the patient.

A medical professional may select to administer fluid directly into a patient's blood vessels for various reasons. For one, fluids administered intravenously are sent directly to the blood stream, resulting in expedited effect of the administered substances contained in the fluid. Rapid administration may be particularly important in a medical emergency, such as when a patient is experiencing a stroke, heart attack, extreme dehydration, or similar emergency conditions. Another reason a medical professional may select to administer fluids intravenously may be to control the precise dosage of substances contained within the administered fluid. For example, a fluid may contain antibiotics, chemotherapy drugs, pain medications, and/or other medications. These substances may be mixed with a solution containing, for example, saline and administered through the patient's blood vessels over time, thus, controlling the amount and duration a particular medication is received. In addition, some medications may not be capable of administration orally, due to enzymes in a patient's mouth and stomach. A medical professional may choose to administer such substances through an IV fluid supply system.

In general, the IV fluid supply bag used to administer fluids intravenously may need to be pressurized to maintain a constant pressure in the IV fluid supply tube fluidly connecting the IV fluid supply bag to the patient. Maintaining constant pressure while administering fluids intravenously prevents air from entering the fluid supply. In addition, applying pressure to the IV fluid supply bag may allow the IV fluid to flow at a consistent rate necessary to maintain a regulated administration of the IV fluid and assimilated substances. Constant pressure of the IV fluid supply may also be necessary to prevent the catheter from clotting due to stagnation. Further, constant pressure of the IV fluid supply may further prevent backflow of the patient's bodily fluids into the IV fluid supply tube.

Current examples require significant manual intervention by a medical professional to maintain a constant pressure in the IV fluid supply. In some examples, a sleeve is wrapped around the IV fluid supply bag which is connected to a manual hand pump. A medical professional may manually inflate the sleeve until the sleeve is at a desired pressure. As IV fluid flows into the patient and the volume of the fluid in the IV fluid supply bag is decreased, the sleeve must be further inflated to maintain the desired pressure. In some instances, an alarm may be triggered when the IV fluid supply pressure is low, alerting a medical professional to further inflate the pressure inducing sleeve. Not only does this require the time and consistent attention of the medical professional, but the patient can further experience alarm fatigue, induced by the constant bombardment of medical device alarms. Further, the accuracy of these pressure regulating methods is also reduced due to the manual nature of the operations and further due to the placement of the pressure monitoring device. Current examples often attach a pressure gauge to the inflatable sleeve, allowing the medical professional to manually monitor the pressure of the air in the sleeve as the sleeve is being inflated. This manual process coupled with the fact that the pressure is only being measured at the sleeve, during inflation, and not in the output fluid supply tube, may lead to inconsistent pressure in the IV fluid supply tube and inconsistent flow of IV fluid.

The various example embodiments described herein utilize various techniques to regulate the IV fluid supply pressure in an IV fluid supply system. For example, in some embodiments, a pump may be fluidly connected to an inflatable bag, sleeve, or other compartment adjacent to the IV fluid supply bag, such that when the pump is enabled, air is pumped into the pressure bag, applying pressure to the IV fluid supply bag. An example embodiment may further include a pressure sensor positioned to measure the pressure within the IV fluid supply tube providing IV fluid to the patient from the IV fluid supply bag. The pressure sensor may provide real-time updates of the IV fluid supply pressure through a communication interface with a controller and/or display device. The controller may complete the feedback loop by altering the output of the pump through a pump communication channel, thus controlling the inflation of the inflatable pressure bag and thus the pressure in the IV fluid supply tube.

By controlling the pressure of the IV fluid supply to the patient through measuring the pressure in the IV fluid supply tube and altering the output of the pump based on the measurement, the example IV fluid supply system may automatically regulate the pressure of the IV fluid supply tube without manual intervention. The various example embodiments of the IV fluid supply system enable continuous real-time updates to the IV fluid supply pressure, thus increasing the accuracy and consistency of the IV fluid flow into the patient. Further, autonomous monitoring of the IV fluid supply pressure unburdens medical professionals from consistent monitoring and manual updating of the pressure applied to an IV fluid supply bag. In addition, the example IV fluid supply system may reduce the number of alarms required to alert medical professionals which unavoidably leads to alarm fatigue in the patient.

As a result of the herein described example embodiments and in some examples, the effectiveness of IV fluid administration by an IV fluid supply system may be greatly improved. In addition, medical professionals may be relieved of manually regulating the pressure of an IV fluid supply system and freed to attend to other important duties.

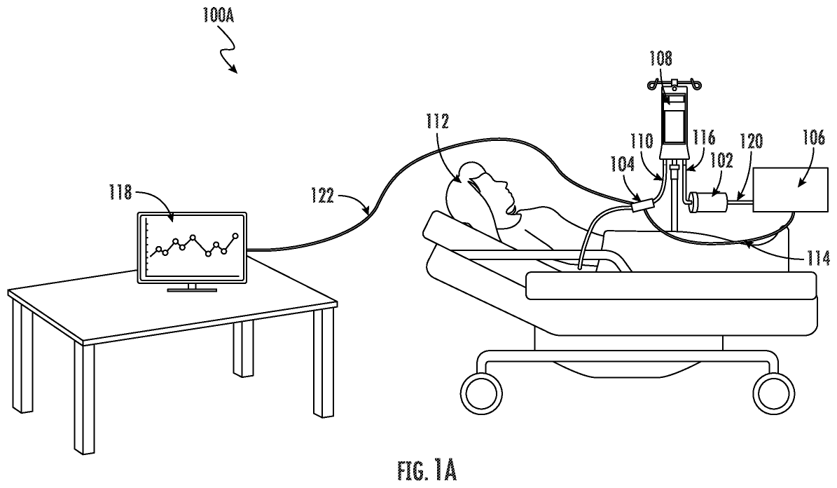

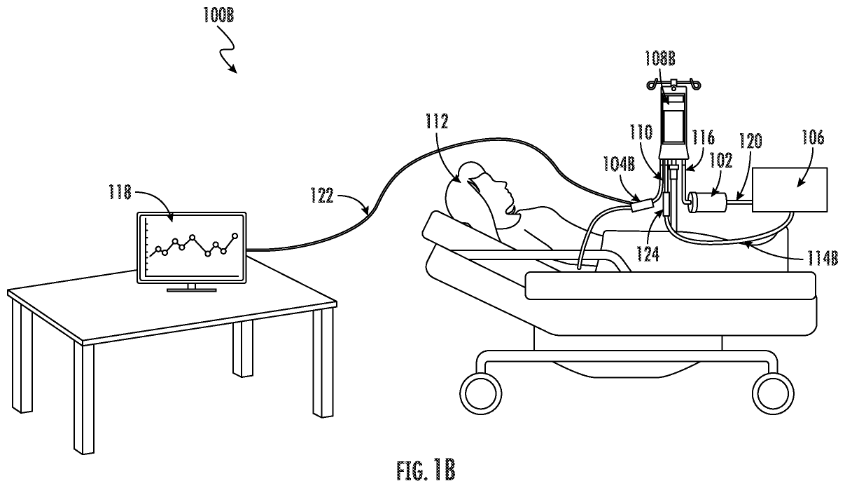

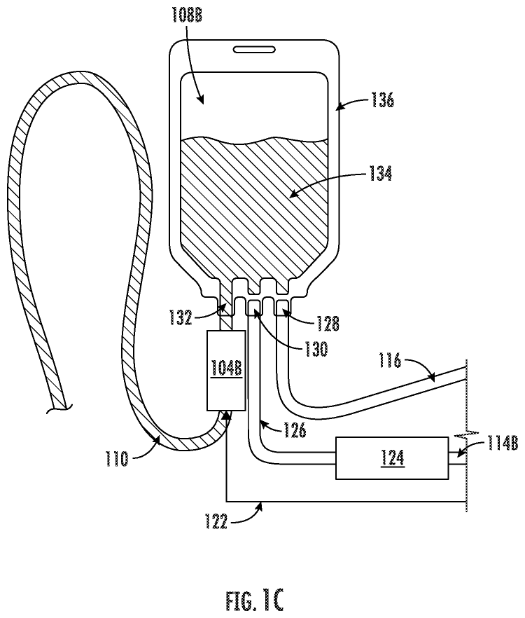

FIG. 1A illustrates an overall system view of an example IV fluid supply system 100A according to one or more embodiments of the present disclosure. As depicted in the example embodiment of FIG. 1A, the example IV fluid supply system 100A includes a pump 102, fluidly connected to an IV fluid supply bag 108 through a pump supply tube 116 and communicatively connected to a controller 106 via a pump communication channel 120. In addition, the controller 106 is further communicatively connected a pressure sensor 104 positioned to detect the pressure within an IV fluid supply tube 110 providing fluid communication between the IV fluid supply bag 108 and a patient 112. Additionally, as further depicted in FIG. 1A, a display device 118 is communicatively connected to the pressure sensor 104 via a display communication channel 122.

Referring now to FIG. 1B, an overall system view of an example IV fluid supply system 100B in accordance with one or more embodiments of the present disclosure is illustrated. In addition, FIG. 1C illustrates a close-up view of the IV fluid supply bag 108B and connected components. As depicted in the example embodiment of FIG. 1B, the example IV fluid supply system 100B includes an additional pressure sensing device, additional pressure sensor 124. In the example IV fluid supply system 100B, the additional pressure sensor 124 is fluidly connected to the pressure bag 136 adjacent to and/or enclosing the IV fluid supply bag 108B. The pressure sensor 124 is further communicatively connected to the controller 106 via the sensor communication channel 114B. The pressure sensor 104B continues to monitor the pressure of the IV fluid in the IV fluid supply tube 110 and transmit pressure measurements in the IV fluid supply tube 110 to the display device 118. The controller 106, in some embodiments, may update the output of the pump 102 based on the pressure measurements of the pressure sensor 104B and/or the additional pressure sensor 124.

Referring now to FIG. 1C, an example IV fluid supply bag 108B and connected components are depicted. As depicted in the example embodiments of FIG. 1C, the example IV fluid supply bag 108B is adjacent to a pressure bag 136, such that in an instance in which the pressure bag 136 is filled with fluid, or otherwise inflated, pressure is applied to the IV fluid supply bag 108B containing IV fluid reservoir 134. The pressure bag 136, in some embodiments, may be fluidly connected to a pump (e.g., pump 102) via an inlet port 128 and a pump supply tube 116. In some embodiments, the IV fluid contained in the IV fluid reservoir 134 may be ejected out of the IV outlet port 132 and into the IV fluid supply tube 110, providing a fluid connection from the IV fluid supply bag 108B to the patient 112. The pressure sensor 104B may be positioned to determine the pressure measurements in the IV fluid supply tube 110. In some embodiments, the pressure measurements captured by the pressure sensor 104B may be transmitted to the display device 118 for display to the patient 112 and/or medical professionals. In some embodiments, additionally, or alternatively, the pressure measurements captured my pressure sensor 104B may be transmitted to the controller 106 through a communication channel, such as the sensor communication channel 114 depicted in FIG. 1A.

As further depicted in FIG. 1C, the example IV fluid supply system 100B includes an additional pressure sensor 124. The additional pressure sensor 124 may, in some embodiments, comprise a pressure sensing device similar to the pressure sensor 104. In some embodiments, the IV fluid supply bag 108B may further include a second outlet port (e.g., pressure outlet port 130) fluidly connecting the pressure sensor bag 136 to the additional pressure sensor 124 through the pressure sensor tube 126. The additional pressure sensor 124 may additionally be communicatively connected to the controller 106 through a sensor communication channel 114B. In some embodiments, the additional pressure sensor 124 may determine the pressure within the pressure bag 136 and transmit the pressure measurements to the controller 106 through the sensor communication channel 114B. In some embodiments, the controller 106 may update the output of the pump 102 based on the pressure in the pressure bag 136 as determined by the additional pressure sensor 124.

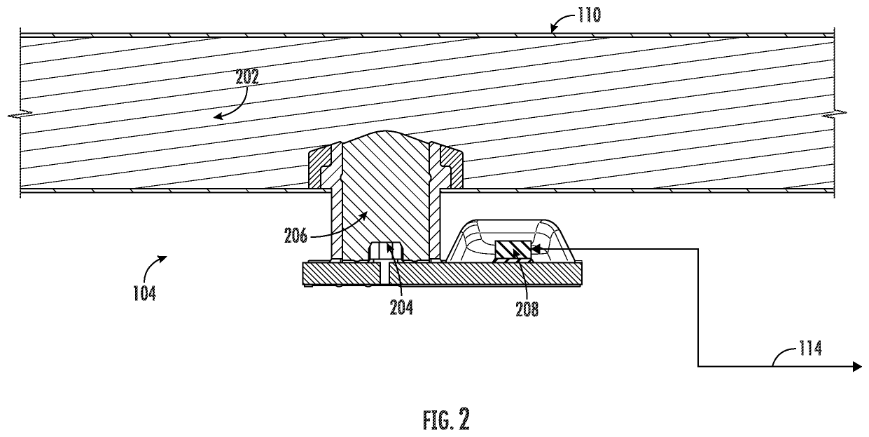

Referring now to FIG. 2, a cross-sectional view of an example pressure sensor 104 within an IV fluid supply system 100A is provided. As depicted in FIG. 2, the pressure sensor 104 includes a pressure sensing diaphragm 204 and a pressure transmitting medium 206 interacting with IV fluid 202 in the IV fluid supply tube 110. The example pressure sensor 104 further includes pressure sensor circuitry 208 communicatively connected to the controller 106 by the sensor communication channel 114.

As illustrated in FIG. 2, the example IV fluid supply system 100A includes a pressure sensor 104. A pressure sensor 104 (104B) may be any electrical, mechanical, and/or electro-mechanical device capable of generating an electrical signal as a function of the pressure imposed by the surrounding environment. As depicted in FIG. 2, the pressure sensor 104 includes a pressure sensing diaphragm 204. In some embodiments, the pressure sensing diaphragm 204 may comprise a semiconductor material. In some embodiments, the pressure sensing diaphragm 204 may contain structures that change properties based on the force applied to the surface of the pressure sensing diaphragm 204. For example, the pressure sensing diaphragm 204 may comprise piezoresistive sensors arranged in a Wheatstone bridge circuit, such that pressure on and deflection of the pressure sensing diaphragm 204 creates a change in resistance in the disposed sensors and an output voltage correlated to the force applied to the surface of the pressure sensing diaphragm 204 from the surrounding environment.

As further illustrated in FIG. 2, the pressure sensor 104 includes a pressure transmitting medium 206. In some embodiments, the pressure sensing diaphragm 204 may be a component of a media-isolated pressure sensor. In a media-isolated pressure sensor (e.g., pressure sensor 104), the pressure sensing diaphragm 204 may be isolated from the measured media (e.g., IV fluid 202) by a structure or substance. In such an embodiment, pressure transmitting medium 206 acts as a coupling mechanism between the IV fluid 202 and the pressure sensing diaphragm 204. The pressure transmitting medium 206 may be a gel or other incompressible material such that any force induced by the IV fluid 202 is propagated through the pressure transmitting medium 206 and to the pressure sensing diaphragm 204 of the pressure sensor 104.

As further illustrated in FIG. 2, the example pressure sensor 104 further includes pressure sensor circuitry 208. In some embodiments, the pressure sensor circuitry 208 may be electrically connected to the pressure sensing diaphragm 204 and may be configured to detect and/or receive the changes in the physical characteristics (e.g., resistance) of the pressure sensing diaphragm 204 due to pressure applied to the surface of the pressure sensing diaphragm 204. The pressure sensor circuitry 208 may, in some embodiments, convert the detected change in resistance into a pressure measurement indicative of the pressure applied by the pressure inducing media (e.g., IV fluid 202) to the pressure transmitting medium 206. In some embodiments, the pressure sensor circuitry 208 may output the pressure reading in analog format while in other embodiments, the pressure sensor circuitry 208 may output the pressure reading in a digital format. In some embodiments, the pressure sensor circuitry 208 may comprise a processor, specially configured field programmable gate array (FPGA), specially programmed application specific integrated circuit (ASIC), trimmable film resistor network, or other similar computing device.

The pressure sensor circuitry 208 may further be configured to transmit the determined pressure measurements utilizing one or more sensor communication channels 114. In some embodiments, the pressure sensor circuitry 208 may communicate with additional components of the IV fluid supply system 100A such as the controller 106 and/or the display device 118. In some embodiments, the pressure sensor circuitry 208 may communicate with such additional components through wireless protocols, for example, IEEE 802.11 Wi-Fi, near field communication (NFC) protocols, Wibree, Bluetooth protocols, wireless universal serial bus (USB) protocols, and/or any other wireless protocol. In some embodiments, the pressure sensor circuitry 208 may communicate with such additional components utilizing standard, wired protocols.

As further illustrated in FIG. 2, the example pressure sensor 104 is positioned such that the pressure transmitting medium 206 is interacting with the IV fluid 202 in the IV fluid supply tube 110, facilitating measurement of pressure applied by the IV fluid 202 within the IV fluid supply tube 110. As illustrated with reference to FIG. 1A, the example IV fluid supply tube 110 provides a fluid connection between the IV fluid supply bag 108 and the patient 112. An IV fluid supply tube 110 may be any tube, conduit, channel, or other similar structure capable of providing fluid communication between components of the IV fluid supply system 100A. Particularly, the IV fluid supply tube 110 may be any conduit transmitting fluid at any portion between the IV fluid supply bag 108 and the patient 112. In some embodiments, the IV fluid supply tube 110 may comprise a biocompatible material such as polyvinyl chloride, polyethylene, thermoplastic elastomers, polypropylene plastic, or other similar material. The IV fluid supply tube 110 wall may provide a sealed conduit, such that the IV fluid 202, or other liquid contained in the IV fluid supply tube 110 may not permeate the IV fluid supply tube 110 wall. In some embodiments, the IV fluid supply tube 110 may be transparent, such that the contents of the IV fluid supply tube 110, such as IV fluid 202, may be visible.

As further illustrated in FIG. 2, the IV fluid supply tube 110 contains IV fluid 202. IV fluid 202 may be any fluid and/or solution intended to be injected into a blood vessel of a patient 112. IV fluid 202 may comprise saline, dextrose in water, colloids, lactated ringers, or other similar solutions. In addition, IV fluid 202 may contain vitamins, minerals, nutrients, medications, and or other substances to be administered to the patient intravenously. In some embodiments, a medical professional may seek to administer the IV fluid 202 at a consistent rate based on the assimilated substances within the IV fluid 202. The rate at which the IV fluid 202 is introduced into the body of the patient 112 may be determined based, at least in part, on the pressure within the IV fluid supply tube 110.

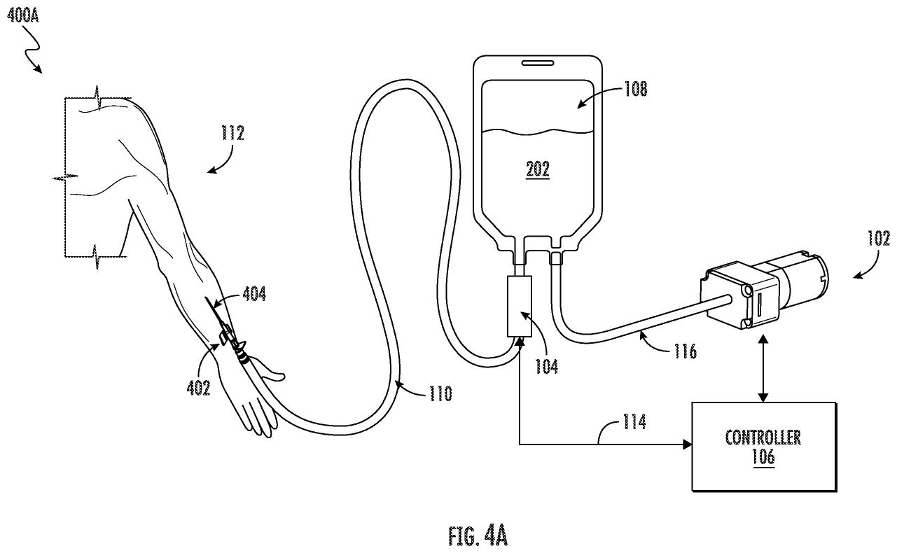

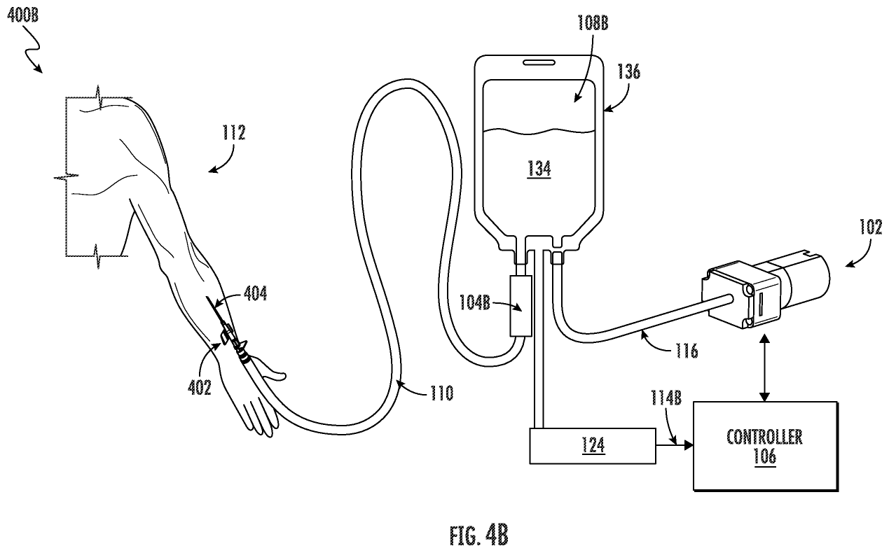

While depicted within the IV fluid supply tube 110, the pressure sensor 104 may be positioned to measure the pressure of the IV fluid 202 anywhere between the IV fluid supply bag 108 and the patient 112. This may be in an IV tube, such as IV fluid supply tube 110, within a pressure transducer (e.g., a disposable pressure transducer or DPT), within a clamp or sampling port, as part of the cannula 402 (as shown in FIG. 4A), in conjunction with a flush valve, at or near a roller clamp, or in conjunction with any other structure or device that provides access to the IV fluid 202 between the IV fluid supply bag 108 and the patient 112.

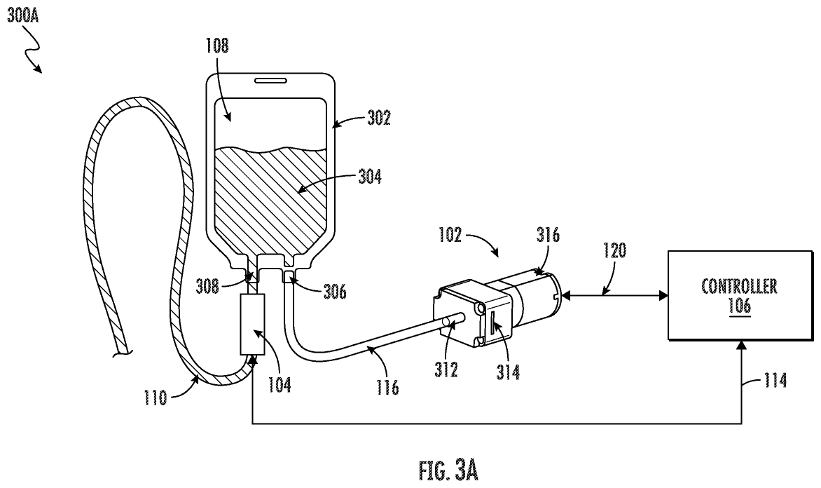

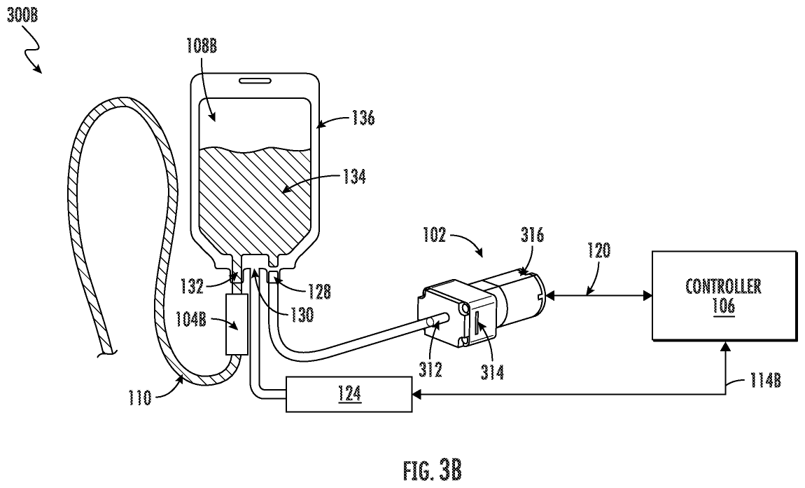

Referring now to FIG. 3A, an example pump 102 of an IV fluid supply system 300A is provided. As shown in FIG. 3A, the example pump 102 is communicatively connected to a controller 106 via a pump communication channel 120. Further, the example pump 102 is fluidly connected to an inlet port 306 of a pressure bag 302 which envelops the IV fluid supply bag 108. As further depicted in FIG. 3A, the IV fluid supply bag 108 is fluidly connected to the IV fluid supply tube 110 through an outlet port 308 on the IV fluid supply bag 108. The outlet port 308 on the IV fluid supply bag 108 allows IV fluid 202 to flow out of the IV fluid supply bag 108, and into the IV fluid supply tube 110. A pressure sensor 104 is further positioned to measure the pressure of the IV fluid 202 within the IV fluid supply tube 110.

As illustrated in FIG. 3A, the IV fluid supply system 100A includes a pump 102. A pump 102 may be any electrical, mechanical, and/or electro-mechanical device that receives air and/or another fluid through one or more inlet ports 306 and ejects, or pumps the fluid out of a pump output port 312. In some embodiments, the pump output port 312 may be fluidly connected to the pump supply tube 116, allowing air and/or another fluid to be pumped from the pump 102 through the pump supply tube 116 and into the pressure bag 302.

In some embodiments, the pump 102 may output fluid at a single pressure level. For example, when the pump 102 is activated, the pump motor 316 may operate at a fixed voltage or single speed, capable of pumping air into a compartment, such as pressure bag 302, until a maximum pressure is reached by the pump 102. In such embodiments, the pump 102 may be activated until a pressure at or near the target IV fluid supply pressure (e.g., 300 millimeters of mercury) is reached, at which point the pump 102 may be deactivated. In an instance in which the IV fluid supply pressure drops below a minimum threshold, the pump 102 may be activated to once again increase the IV fluid supply pressure to the determined target IV fluid supply pressure.

In some embodiments, the pump 102 may output fluid at variable rates. For example, the pump 102 may operate at different voltages and/or pump air at different speeds. In such embodiments, the speed of the output air may be adjusted according to the measured IV fluid supply pressure. Adjusting the output of the pump 102 may include activating and deactivating the pump, and/or varying the speed/power at which air is output. Although illustrated in FIG. 3A as a pump motor 316, the air and/or other fluid may be inlet and ejected using a electromagnet mechanism and/or any other mechanism to pump air and/or other fluid out of the pump output port 312.

In some embodiments, the pump 102 may receive configuration parameters and/or commands via a communication interface through a communication channel such as pump communication channel 120. In addition, in some embodiments, the pump 102 may be activated and/or deactivated through the pump communication channel 120. Commands may include instructions to increase/decrease the speed of the pump 102, start/stop the pump 102, update the target IV fluid supply pressure and/or minimum IV fluid supply threshold, and/or other similar commands. In addition, the pump 102, in some embodiments, may communicate status and/or real-time pressure settings and measurements.

As further illustrated in FIG. 3A, the example IV fluid supply system 300A includes a pump supply tube 116 fluidly connecting the pump 102 to the inlet port 306 of the IV fluid supply bag 108. The pump supply tube 116 may comprise a biocompatible material such as polyvinyl chloride, polyethylene, thermoplastic elastomers, polypropylene plastic, or other similar material, providing a conduit for fluid to flow from the pump 102 and into the pressure bag 302 of the IV fluid supply bag 108.

As further illustrated in FIG. 3A, the example IV fluid supply system 300A includes a IV fluid supply bag 108. As depicted in FIG. 3A, the example IV fluid supply bag 108 (108B) includes an interior IV fluid reservoir 304 holding a supply of IV fluid 202. An IV fluid reservoir 304 may be any bag, reservoir, container, receptacle, and/or the like capable of holding a supply of IV fluid or other similar fluid. In some embodiments, the IV fluid reservoir 304 may comprise vinyl or soft plastic. The IV fluid contained within the IV fluid supply bag 108 may be intended to enter into a blood vessel of the patient 112 as described in relation to FIG. 2. As depicted in FIG. 3A, the IV fluid reservoir 304 is fluidly connected to the IV fluid supply tube 110 through the outlet port 308. The outlet port 308 provides a direct fluid connection from the IV fluid reservoir 304 to the IV fluid supply tube 110. In some embodiments, the IV fluid reservoir 304 may be pressurized to prevent air from entering the IV fluid 202 supply, allow the IV fluid 202 to flow at a consistent rate, prevent clotting due to stagnation, and prevent backflow of the patient's bodily fluids into the IV fluid supply tube 110.

In the example embodiment depicted in FIG. 3A, the IV fluid supply bag 108 includes an interior IV fluid reservoir 304 containing a supply of IV fluid 202, surrounded by a pressure bag 302. A pressure bag 302 (see also pressure bag 136) may be any attached compartment, sleeve, or other container that may receive air and/or other fluid into the pressure bag 302 compartment through the inlet port 306. In some embodiments, the pressure bag 302 may comprise vinyl or soft plastic. In some embodiments, the pressure bag 302 may be attached to the IV fluid reservoir 304 (see also IV fluid reservoir 134) and substantially encase the IV fluid reservoir 304, such that when air and/or another fluid are pumped into the pressure bag 302, the pressure bag 302 inflates, increasing the pressure in the IV fluid reservoir 304 and subsequently the pressure of the IV fluid 202 ejected from the IV fluid reservoir 304 and into the IV fluid supply tube 110. In some embodiments, the pressure bag 302 may be a detached sleeve placed around the IV fluid reservoir 304 similarly applying pressure to the IV fluid reservoir 304 when inflated with air and/or another fluid. As depicted in FIG. 3A, the pressure bag 302 includes a direct fluid connection to the pump supply tube 116 through the inlet port 306. Such a connection enables the pressure bag 302 to be inflated by an inflating device (e.g., pump 102) through the inlet port 306.

Referring now to FIG. 3B, an IV fluid supply system 300B is provided. As shown in FIG. 3B, the example IV fluid supply system 300B includes an additional pressure sensor 124 fluidly connected to the pressure bag 136 via a pressure outlet port 130 on the IV fluid supply bag 108B. The additional pressure sensor 124 is further communicatively connected to the controller 106 via the sensor communication channel 114B. The controller 106, in some embodiments, may update the output of the pump 102 based on the pressure measurements of the pressure sensor 104B and/or the additional pressure sensor 124.

Referring now to FIG. 4A, an example IV fluid supply system 400A transmitting IV fluid 202 to a patient 112 is provided. As shown in FIG. 4A, the example IV fluid supply system 400A additionally includes a cannula 402 fluidly connected to the IV fluid supply tube 110 at one end and to the blood vessel of the patient 112 through a catheter 404 at an opposite end. As depicted in FIG. 4A, in some embodiments, the IV fluid supply system may further include a display device 118 communicatively connected to the pressure sensor 104 and/or controller 106 via a display communication channel 122.

As illustrated in FIG. 4A, the example IV fluid supply system 400A includes an IV fluid supply tube 110 fluidly connecting the patient 112 to the IV fluid 202 flowing from the IV fluid reservoir 304. Maintaining at least a minimum pressure in the IV fluid supply tube 110 provides various benefits including preventing air from entering the IV fluid supply tube 110 from the IV fluid reservoir 304, allowing the IV fluid 202 to flow at a consistent rate, preventing clotting due to stagnation, and preventing backflow of the patient's 112 bodily fluids into the IV fluid supply tube 110. To maintain consistent pressure in the IV fluid supply tube 110, a pressure sensor 104 (described in relation to FIG. 2) is positioned to measure the pressure in the IV fluid supply tube 110 between the IV fluid reservoir 304 and the patient 112. Measurements determined by the pressure sensor 104 may be transmitted to the controller 106 and used to adjust the output of the pump 102. Adjusting the output of the pump 102 may include activating/deactivating the pump 102 and/or adjusting the speed and/or output power of the pump 102.

As further illustrated in FIG. 4A, the example IV fluid supply system 400A includes a cannula 402. The cannula 402 may be any device or structure comprising a thin tube (e.g., catheter 404) that may be inserted into a patient's 112 body cavity, for example, a blood vessel, to provide a fluid connection from the IV fluid supply tube 110 to the patient's 112 body cavity. In some embodiments, the cannula 402 may comprise a body or structure to define a conduit providing a fluid connection between the IV fluid supply tube 110 and the patient's 112 body cavity. The cannula 402 may further provide structures and/or features to support a catheter 404 and provide convenience in inserting the catheter 404 into the patient's 112 blood vessel or other cavity. In some embodiments (not shown) the cannula 402 may further include additional structures providing connection points for administering medications and drawing blood; structures for preventing back flow of bodily fluids; and/or the like. The example cannula 402 may further include a leak-free connection to the IV fluid supply tube 110. The leak-free connection may be any connecting structure that fluidly connects the IV fluid supply tube 110 to the cannula 402 such that fluid leaks are substantially limited. In some embodiments, the leak-free connection may comprise a Luer connector, Luer-lock fitting, Luer taper, or other similar leak free connection.

As further depicted in FIG. 4A, the example cannula 402 further includes a catheter 404. The catheter 404 may be any thin tube comprised of medical grade materials that may be inserted into a body cavity of a patient 112 (e.g., a blood vessel) and provide fluid communication, for example through a narrow conduit, to the body cavity of the patient 112 from outside the body of the patient 112. In some embodiments, the catheter 404 may further include a pointed tip to pierce the skin of the patient 112 and insert the end of the catheter 404 into the body cavity of the patient 112. In some embodiments, the catheter 404 may be attached to the cannula 402, such that the IV fluid supply tube 110 may be fluidly coupled with the catheter 404, such that a fluid, for example IV fluid 202, may flow from the IV fluid supply tube 110, through the cannula 402 and the catheter 404 and into the body cavity of the patient 112. Maintaining an IV fluid supply pressure above a determined minimum threshold, prevents bodily fluids of the patient 112 from entering into the catheter 404 and into the IV fluid supply tube 110. Further, maintaining a minimum IV fluid supply pressure allows the IV fluid 202 to flow at a consistent rate and prevents clotting due to stagnation.

As further depicted in FIG. 4A, the example IV fluid supply system 400A of FIG. 4A, includes a display device 118 communicatively connected to the pressure sensor 104 and/or the controller 106 through the display communication channel 122. A display device 118 may be any monitor, screen, television, mobile device, tablet, and/or other display utilized to present recorded and/or predicted information to a patient 112 and medical professionals. For example, a display device 118 may depict the pressure within the IV fluid supply tube 110, as recorded by the pressure sensor 104, as a function of time. In some embodiments, the display device 118 may update a user interface based on data received directly from the pressure sensor 104. In some embodiments, the display device 118 may update a user interface based on data received from the controller 106. Updates may be transmitted via the display communication channel 122 via wired communication protocols and/or wireless communication protocols.

Referring now to FIG. 4B, an example IV fluid supply system 400B transmitting IV fluid 134 to a patient 112 is provided. As shown in FIG. 4B, the example IV fluid supply system 400B includes an additional pressure sensor 124 fluidly connected to the pressure bag 136 adjacent to the IV fluid supply bag 108B. The additional pressure sensor 124 is further communicatively connected to the controller 106 via the sensor communication channel 114B. The pressure sensor 104B continues to monitor the pressure of the IV fluid 134 in the IV fluid supply tube 110 providing IV fluid 134 to the patient 112. The controller 106, in some embodiments, may update the output of the pump 102 based on the pressure measurements of the pressure sensor 104B and/or the additional pressure sensor 124.

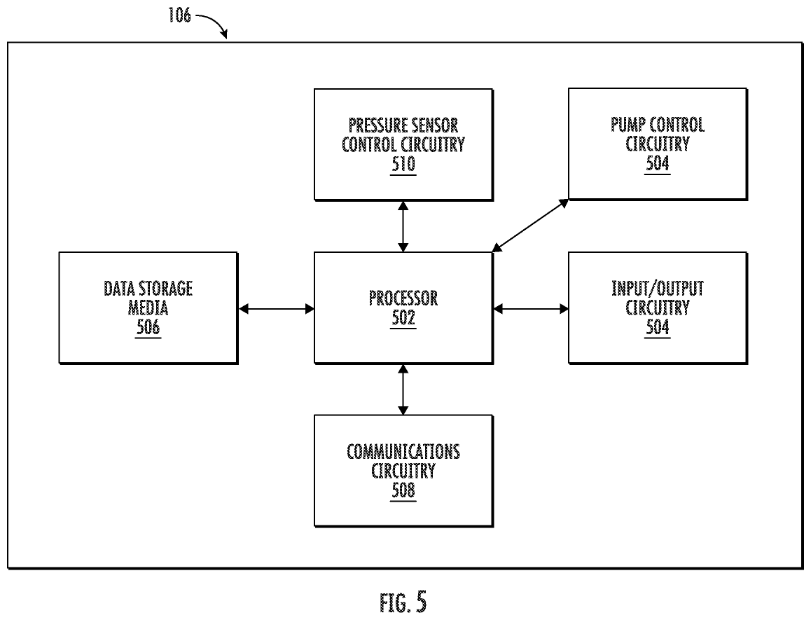

FIG. 5 illustrates an example controller 106 in accordance with at least some example embodiments of the present disclosure. The controller 106 includes processor 502, input/output circuitry 504, data storage media 506, communications circuitry 508, pressure sensor control circuitry 510, and pump control circuitry 512. In some embodiments, the controller 106 is configured, using one or more of the sets of circuitry 502, 504, 506, 508, 510, and/or 512, to execute and perform the operations described herein.

Although components are described with respect to functional limitations, it should be understood that the particular implementations necessarily include the use of particular computing hardware. It should also be understood that in some embodiments certain of the components described herein include similar or common hardware. For example, two sets of circuitry may both leverage use of the same processor(s), network interface(s), storage medium(s), and/or the like, to perform their associated functions, such that duplicate hardware is not required for each set of circuitry. The user of the term “circuitry” as used herein with respect to components of the apparatuses described herein should therefore be understood to include particular hardware configured to perform the functions associated with the particular circuitry as described herein.

Particularly, the term “circuitry” should be understood broadly to include hardware and, in some embodiments, software for configuring the hardware. For example, in some embodiments, “circuitry” includes processing circuitry, storage media, network interfaces, input/output devices, and/or the like. Alternatively or additionally, in some embodiments, other elements of the controller 106 provide or supplement the functionality of other particular sets of circuitry. For example, the processor 502 in some embodiments provides processing functionality to any of the sets of circuitry, the data storage media 506 provides storage functionality to any of the sets of circuitry, the communications circuitry 508 provides network interface functionality to any of the sets of circuitry, and/or the like.

In some embodiments, the processor 502 (and/or co-processor or any other processing circuitry assisting or otherwise associated with the processor) is/are in communication with the data storage media 506 via a bus for passing information among components of the controller 106. In some embodiments, for example, the data storage media 506 is non-transitory and may include, for example, one or more volatile and/or non-volatile memories. In other words, for example, the data storage media 506 in some embodiments includes or embodies an electronic storage device (e.g., a computer readable storage medium). In some embodiments, the data storage media 506 is configured to store information, data, content, applications, instructions, or the like, for enabling the controller 106 to carry out various functions in accordance with example embodiments of the present disclosure.

The processor 502 may be embodied in a number of different ways. For example, in some example embodiments, the processor 502 includes one or more processing devices configured to perform independently. Additionally or alternatively, in some embodiments, the processor 502 includes one or more processor(s) configured in tandem via a bus to enable independent execution of instructions, pipelining, and/or multithreading. The use of the terms “processor” and “processing circuitry” should be understood to include a single core processor, a multi-core processor, multiple processors internal to the controller 106, and/or one or more remote or “cloud” processor(s) external to the controller 106.

In an example embodiment, the processor 502 is configured to execute instructions stored in the data storage media 506 or otherwise accessible to the processor. Alternatively or additionally, the processor 502 in some embodiments is configured to execute hard-coded functionality. As such, whether configured by hardware or software methods, or by a combination thereof, the processor 502 represents an entity (e.g., physically embodied in circuitry) capable of performing operations according to an embodiment of the present disclosure while configured accordingly. Alternatively or additionally, as another example in some example embodiments, when the processor 502 is embodied as an executor of software instructions, the instructions specifically configure the processor 502 to perform the algorithms embodied in the specific operations described herein when such instructions are executed.

As one particular example embodiment, the processor 502 is configured to perform various operations associated with regulating the IV fluid supply pressure in an IV fluid supply system 100A, 100B, 300A, 300B, 400A, 400B. In some embodiments, the processor 502 includes hardware, software, firmware, and/or a combination thereof, that receives a pressure measurement of a plurality of pressure measurements from a pressure sensor 104, wherein the pressure sensor 104 is positioned to measure the pressure within an IV fluid supply tube 110, and wherein the IV fluid supply tube 110 is fluidly connected to an outlet port 308 of an IV fluid supply bag 108. Additionally or alternatively, in some embodiments, the processor 502 includes hardware, software, firmware, and/or a combination thereof, that determines an output of a pump 102, fluidly connected to an inlet port 306 of a pressure bag 302, based at least in part on the pressure measurement received from the pressure sensor 104, wherein the pressure bag 302 is adjacent to the IV fluid supply bag 108 containing the IV fluid 202, and wherein in an instance in which a fluid is pumped into the inlet port 306 of the pressure bag 302, pressure is applied to the IV fluid supply bag 108, such that, IV fluid 202 is ejected from an outlet port 308 into the IV fluid supply tube 110. Additionally or alternatively, in some embodiments, the processor 502 includes hardware, software, firmware, and/or a combination thereof, that alters the output of the pump 102 based at least in part on the pressure measurement received from the pressure sensor 104.

In some embodiments, the controller 106 includes input/output circuitry 504 that provides output to the user and, in some embodiments, to receive an indication of a user input. In some embodiments, the input/output circuitry 504 is in communication with the processor 502 to provide such functionality. The input/output circuitry 504 may comprise one or more user interface(s) (e.g., user interface) and in some embodiments includes a display that comprises the interface(s) rendered as a web user interface, an application user interface, a user device, a backend system, or the like. The processor 502 and/or input/output circuitry 504 comprising the processor may be configured to control one or more functions of one or more user interface elements through computer program instructions (e.g., software and/or firmware) stored on a memory accessible to the processor (e.g., data storage media 506, and/or the like). In some embodiments, the input/output circuitry 504 includes or utilizes a user-facing application to provide input/output functionality to a client device and/or other display associated with a user.

In some embodiments, the controller 106 includes communications circuitry 508. The communications circuitry 508 includes any means such as a device or circuitry embodied in either hardware or a combination of hardware and software that is configured to receive and/or transmit data from/to a network and/or any other device, circuitry, or module in communication with the controller 106. In this regard, the communications circuitry 508 includes, for example in some embodiments, a network interface for enabling communications with a wired or wireless communications network. Additionally or alternatively in some embodiments, the communications circuitry 508 includes one or more network interface card(s), antenna(s), bus(es), switch(es), router(s), modem(s), and supporting hardware, firmware, and/or software, or any other device suitable for enabling communications via one or more communications network(s). Additionally or alternatively, the communications circuitry 508 includes circuitry for interacting with the antenna(s) and/or other hardware or software to cause transmission of signals via the antenna(s) or to handle receipt of signals received via the antenna(s). In some embodiments, the communications circuitry 508 enables transmission to and/or receipt of data from a client device in communication with the controller 106.

The pressure sensor control circuitry 510 includes hardware, software, firmware, and/or a combination thereof, that supports various functionality associated with configuring and/or communicating with a pressure sensor 104. For example, in some embodiments, the pressure sensor control circuitry 510 includes hardware, software, firmware, and/or a combination thereof to communicate with the pressure sensor 104 according to an established protocol to provide appropriate configuration and/or calibration parameters to receive accurate data representing the environmental pressure of the measured environment. Additionally or alternatively, in some embodiments, the pressure sensor control circuitry 510 includes hardware, software, firmware, and/or a combination thereof, to convert voltages and/or other output data returned from the pressure sensor 104 into pressure readings. Additionally or alternatively, in some embodiments, the pressure sensor control circuitry 510 includes hardware, software, firmware, and/or a combination thereof, that reconfigures and/or reinitializes the pressure sensor 104 based on received pressure data. In some embodiments, the pressure sensor control circuitry 510 includes a separate processor, specially configured field programmable gate array (FPGA), or a specially programmed application specific integrated circuit (ASIC).

The pump control circuitry 512 includes hardware, software, firmware, and/or a combination thereof, that supports various functionality associated with configuring and/or communicating with a pump 102. For example, in some embodiments, the pump control circuitry 512 includes hardware, software, firmware, and/or a combination thereof to communicate with the pump 102 according to an established protocol to provide appropriate configuration and/or calibration parameters, including, but not limited to the output power of the pump. Additionally or alternatively, in some embodiments, the pump control circuitry 512 includes hardware, software, firmware, and/or a combination thereof, to receive real-time configuration data and/or settings associated with the current operating condition of the pump 102. Additionally or alternatively, in some embodiments, the pump control circuitry 512 includes hardware, software, firmware, and/or a combination thereof, that enables and/or disables power to the pump 102. For example, in some embodiments, the pump control circuitry 512 may enable the controller 106 to withhold and restore power to the pump 102, effectively activating and deactivating the pump 102. In some embodiments, the pump control circuitry 512 includes a separate processor, specially configured field programmable gate array (FPGA), or a specially programmed application specific integrated circuit (ASIC).

Additionally or alternatively, in some embodiments, one or more of the sets of circuitry 502-512 are combinable. Additionally or alternatively, in some embodiments, one or more of the sets of circuitry perform some or all of the functionality described associated with another component. For example, in some embodiments, one or more sets of circuitry 502-512 are combined into a single module embodied in hardware, software, firmware, and/or a combination thereof. Similarly, in some embodiments, one or more of the sets of circuitry, for example pressure sensor control circuitry 510, and/or pump control circuitry 512, is/are combined such that the processor 502 performs one or more of the operations described above with respect to each of these circuitry individually.

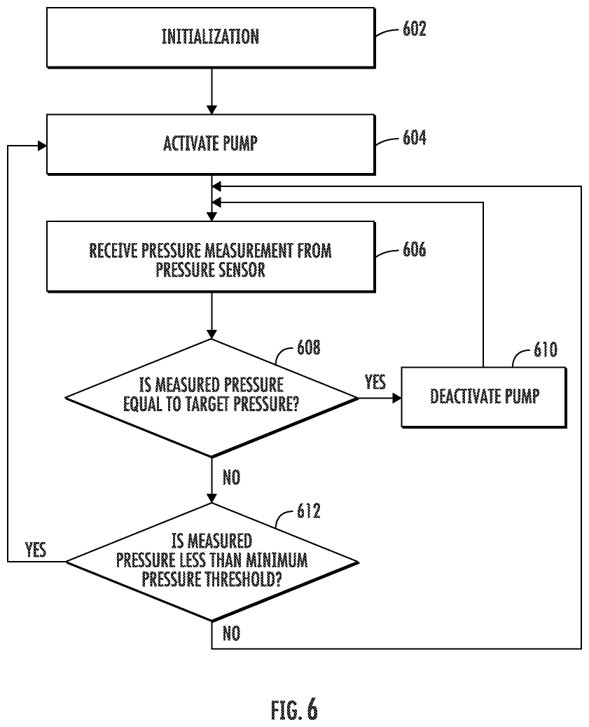

Referring now to FIG. 6, a flowchart illustrating an example process 600 for regulating the IV fluid supply pressure in an example IV fluid supply system (e.g., IV fluid supply system 100A, 100B, 300A, 300B, 400A, 400B) is provided. As depicted in FIG. 6, the process 600 begins at step 602 with initialization of the IV fluid supply system. In some embodiments, initialization may include calibration, testing, and initial configuration of the pressure sensor 104. Calibration of the pressure sensor 104 may include a determination of values required to convert the measured change in physical properties of the pressure sensing diaphragm 204 to a real-world pressure, for the specific environment. Initialization of the pressure sensor 104 may include configuration of the rate and method by which pressure measurements are provided to the controller 106. Further initialization may include calibration, testing, and initial configuration of the pump 102. In addition, the display device 118 may further require an initial configuration. The initialization step 602 may further include configuration of controller 106 parameters, such as the target IV fluid supply pressure, the minimum pressure threshold, and similar controller 106 parameters. Such controller 106 parameters may be determined based on medical and/or other national standards, or may be input by a medical professional during the initialization step 602. The frequency, format, and method of receipt of pressure measurements from the pressure sensor 104 and real-time conditions of the pump 102, may also be configured during the initialization step 602.

The process 600 continues at step 604 when the pump 102 is activated. In some embodiments, the output of the pump 102 may be regulated by the controller 106. One such method of regulating the output of the pump 102 may be to provide power to the motor (e.g., pump motor 316) or other device responsible for inletting air and/or other fluids and injecting the fluid into the pressure bag 302 through the pump supply tube 116. In such an instance, the input voltage, motor speed, and/or speed of another pumping mechanism remains constant and air and/or another fluid is pumped into the pressure bag 302 at a constant rate. In some embodiments, regulating the output of the pump may include adjusting the input voltage and/or motor speed of the pump 102. By adjusting the input voltage and/or motor speed of the pump 102, the maximum pressure and/or rate of changing pressure may be controlled at a more precise level.

The process 600 continues at step 606 when the controller 106 receives pressure measurements from the pressure sensor 104. In some embodiments, the controller 106 may receive output voltages from the pressure sensor 104 representative of the environment pressure measured within the IV fluid supply tube 110. In some embodiments, the pressure sensor 104 may convert the measured change in voltage and/or resistance of the pressure sensing diaphragm 204 to a pressure before transmitting the measurements to the controller 106. In some embodiments, the controller 106 may configure the pressure sensor 104 during the initialization step 602 to transmit pressure measurements at a regular interval. In some embodiments, the controller 106 may request a pressure measurement from the pressure sensor 104 at regular intervals, and/or when triggered by some external event.

The process 600 continues at step 608 when the controller 106 determines if the measured pressure is equal to the target IV fluid supply pressure. The target IV fluid supply pressure may be predetermined based on medical standards, for example, 300 millimeters of mercury. In some embodiments, the target IV fluid supply pressure may be input by a medical professional during the initialization step 602. In some embodiments, the target IV fluid supply pressure may be adjusted in real-time based on the blood pressure of the patient 112, and/or based on changes in desired flow during the administration of the IV fluid 202. In some embodiments, the measure pressure may be accumulated and averaged over a period of time to limit the effect of anomalous values. If the measured pressure is greater than or equal to the target IV fluid supply value, the process 600 will continue to step 610. However, if the measured pressure is less than the target IV fluid supply pressure, the process 600 will continue to step 612.

At step 610, the pump 102 is deactivated. In some embodiments, in an instance in which it is determined that the measured pressure is greater than or equal to the target IV fluid supply pressure, the output of the pump 102 may be regulated by the controller 106. In some embodiments, regulating the output of the pump 102 may include deactivating the pump. In some embodiments, the controller 106 may deactivate the pump 102 by disconnecting the power source from the pump 102. In some embodiments, the controller 106 may configure a power source to cease distributing power to the pump 102. In some embodiments, the controller 106 may transmit one or more control commands, configuring the pump 102 to disconnect the power source. In addition, in some embodiments, the controller 106 may adjust the output of the pump 102 by varying the speed of the output airflow from the pump 102. In some embodiments, the controller 106 may vary the input voltage of provided to the pump 102. In some embodiments, the controller 106 may transmit one or more control commands, configuring the pump 102 to vary the output airflow of the pump 102.

At step 612, the controller 106 determines if the measured pressure is less than a minimum pressure threshold. A minimum pressure threshold may be predetermined based on medical standards, for example, 280 millimeters of mercury. In some embodiments, the minimum pressure threshold may be input by a medical professional during the initialization step 602. In some embodiments, the minimum pressure threshold may be adjusted in real-time based on the blood pressure of the patient 112, and/or based on changes in desired flow during the administration of the IV fluid 202. An IV fluid supply pressure that drops below the minimum pressure threshold may lead to air entering the IV fluid supply tube 110, inconsistent flow rate of the IV fluid 202, clotting due to stagnation, and/or backflow of the patient's 112 bodily fluids into the IV fluid supply tube 110. In an instance in which the measured pressure is less than the minimum pressure threshold, the process continues at step 604 in which the pump 102 is activated to increase the pressure applied to the IV fluid reservoir 304. In an instance in which the measured pressure is greater than or equal to the minimum threshold, the process continues at step 606, in which the controller 106 awaits or requests transmission of an updated pressure sensor 104 measurement.

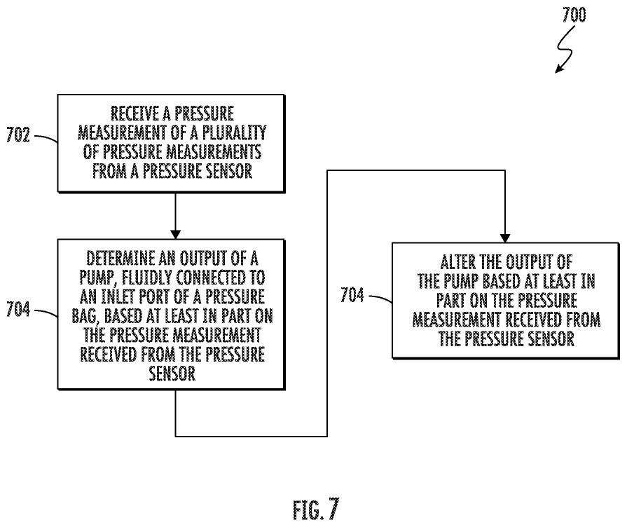

Referring now to FIG. 7, an example flow diagram illustrating an example method 700 for regulating the IV fluid supply pressure in an IV fluid supply system (e.g., IV fluid supply system 100A, 100B, 300A, 300B, 400A, 400B) is illustrated, in accordance with some embodiments of the present disclosure. The example method 700 begins at block 702 when a controller 106 receives a pressure measurement of a plurality of pressure measurements from a pressure sensor 104. As described in relation to FIG. 6, a controller 106 may, in some embodiments, configure a pressure sensor 104 to transmit pressure measurements at a regular interval, for example, every 2 seconds. In some embodiments, the controller 106 may request a pressure measurement at a regular interval and/or based on another triggering event (e.g., user request). In addition, in some embodiments, the pressure sensor 104 may convert the measured change in physical properties of the pressure sensing diaphragm 204 to real-world pressure measurements previous to transmitting the measured values to the controller 106. In some embodiments, the controller 106 may convert the measured change in physical properties of the pressure sensing diaphragm 204 upon receipt.

At block 704, the controller 106 determines an output of the pump 102, fluidly connected to the inlet port 306 of a pressure bag 302, based at least in part on the pressure measurement received from the pressure sensor 104. As further described in relation to FIG. 6, the controller 106 may determine the output of the pump 102 based on the pressure measurements returned by the pressure sensor 104. As depicted in the flowchart of FIG. 6, in some embodiments, a controller 106 may compare the measured pressure to a pre-determined target IV fluid supply pressure. In an instance in which the measured pressure is equal to or greater than the target IV fluid supply pressure, the controller 106 may deactivate the pump 102 or otherwise reduce the output speed and/or power of the pump 102. Alternatively, in an instance in which the measured pressure is less than a minimum pressure threshold, the controller 106 may determine to activate the pump 102 or otherwise increase the output speed and/or power of the pump 102, thus causing more fluid to be pumped into the pressure bag 302 and the IV fluid supply pressure to be increased.

At block 706, the controller 106 alters the output of the pump 102 based at least in part on the pressure measurement received from the pressure sensor 104. Once the controller 106 has determined the output of the pump 102 based on the measurements from the pressure sensor 104, in some embodiments, the controller 106 may configure the pump 102 to output fluid based on the pressure measurements. In some embodiments, the controller 106 may provide the power source to the pump 102. In some embodiments, the controller 106 may configure the power source and/or a device controlling the power provided to the pump 102. In such embodiments, the controller 106 may manipulate the power source to the pump 102 to obtain the desired pump 102 output. For example, the controller 106 may configure the power source to provide power output at a maximum level to the pump 102, thus activating the pump 102 at full power. Additionally, in some embodiments, the controller 106 may vary the output power of the power source, thus varying the speed and/or force with which the pump 102 pumps air or other fluids into the pressure bag 302.

In some embodiments, the controller 106 may communicate with the pump 102 through a wireless or wired protocol in order to alter the output of the pump 102. For example, the controller 106 may send one or more commands to deactivate or turn off the pump 102. Alternatively, the controller 106 may send one or more commands to activate or turn on the pump 102 at full power. Further, the controller 106, in some embodiments, may send command messages to vary the output speed and force of the pump 102 based on the measurements provided by the pressure sensor 104.

Many modifications and other embodiments of the inventions set forth herein will come to mind to one skilled in the art to which these inventions pertain having the benefit of teachings presented in the foregoing descriptions and the associated drawings. Although the figures only show certain components of the apparatus and systems described herein, it is understood that various other components may be used in conjunction with the system. Therefore, it is to be understood that the inventions are not to be limited to the specific embodiments disclosed and that modifications and other embodiments are intended to be included within the scope of the appended claims. Moreover, the steps in the method described above may not necessarily occur in the order depicted in the accompanying diagrams, and in some cases one or more of the steps depicted may occur substantially simultaneously, or additional steps may be involved. Although specific terms are employed herein, they are used in a generic and descriptive sense only and not for purposes of limitation.

While various embodiments in accordance with the principles disclosed herein have been shown and described above, modifications thereof may be made by one skilled in the art without departing from the spirit and the teachings of the disclosure. The embodiments described herein are representative only and are not intended to be limiting. Many variations, combinations, and modifications are possible and are within the scope of the disclosure. Alternative embodiments that result from combining, integrating, and/or omitting features of the embodiment(s) are also within the scope of the disclosure. Accordingly, the scope of protection is not limited by the description set out above.

Additionally, the section headings used herein are provided for consistency with the suggestions under 37 C.F.R. 1.77 or to otherwise provide organizational cues. These headings shall not limit or characterize the invention(s) set out in any claims that may issue from this disclosure.

Use of broader terms such as “comprises,” “includes,” and “having” should be understood to provide support for narrower terms such as “consisting of,” “consisting essentially of,” and “comprised substantially of” Use of the terms “optionally,” “may,” “might,” “possibly,” and the like with respect to any element of an embodiment means that the element is not required, or alternatively, the element is required, both alternatives being within the scope of the embodiment(s). Also, references to examples are merely provided for illustrative purposes, and are not intended to be exclusive.

What is claimed is:

1. An intravenous fluid supply system comprising:
an intravenous fluid supply tube in fluid connection with an outlet port of an intravenous fluid supply bag containing an intravenous fluid,
wherein the intravenous fluid is ejected into the intravenous fluid supply tube upon the application of a pressure on the intravenous fluid supply bag;
a pump fluidly connected to an inlet port of a pressure bag,
wherein in an instance in which the pressure bag is adjacent to the intravenous fluid supply bag, and in an instance in which a fluid is pumped into the inlet port of the pressure bag by the pump, pressure is applied to the adjacent intravenous fluid supply bag;
a pressure sensor positioned to measure a pressure within the intravenous fluid supply tube; and
a controller communicatively connected to the pressure sensor and the pump, wherein the controller regulates an output of the pump based at least in part on one or more measurements of the pressure sensor.

2. The intravenous fluid supply system of claim 1, wherein the intravenous fluid supply tube is fluidly connected to a blood vessel of a patient and wherein the pressure sensor measures the pressure of the intravenous fluid in the intravenous fluid supply tube between the intravenous fluid supply bag and the blood vessel of the patient.

3. The intravenous fluid supply system of claim 2, wherein the controller regulates the output of the pump such that the pressure of the intravenous fluid in the intravenous fluid supply tube is greater than a blood pressure of the patient.

4. The intravenous fluid supply system of claim 2, wherein a cannula is inserted into the blood vessel of the patient, and the pressure sensor measures the pressure in the intravenous fluid supply tube between the patient and the cannula.

5. The intravenous fluid supply system of claim 1, wherein the pressure sensor measures a pressure measurement of the intravenous fluid in the intravenous fluid supply tube and transmits the pressure measurement to the controller.

6. The intravenous fluid supply system of claim 1, wherein in an instance in which the pressure measurement drops below a minimum pressure threshold, the controller adjusts the output of the pump.

7. The intravenous fluid supply system of claim 6, wherein the minimum pressure threshold is between 275 and 285 millimeters of mercury and the pressure of the intravenous fluid in the intravenous fluid supply tube is maintained between 275 millimeters of mercury and 310 millimeters of mercury.

8. The intravenous fluid supply system of claim 1, wherein regulating the output of the pump comprises activating and deactivating the pump.

9. The intravenous fluid supply system of claim 1, wherein the controller transmits the one or more measurements of the pressure sensor to a display device.

10. The intravenous fluid supply system of claim 1, further comprising an additional pressure sensor, wherein the additional pressure sensor is fluidly connected to the pressure bag through a second outlet port, wherein the additional pressure sensor is communicatively connected to the controller, and wherein the controller regulates the output of the pump based at least in part on one or more measurements of the additional pressure sensor.

11. A method for regulating a pressure of an intravenous fluid supply, the method comprising:
receiving a pressure measurement of a plurality of pressure measurements from a pressure sensor,
wherein the pressure sensor is positioned to measure the pressure within an intravenous fluid supply tube, and
wherein the intravenous fluid supply tube is fluidly connected to an outlet port of an intravenous fluid supply bag;
determining an output of a pump, fluidly connected to an inlet port of a pressure bag, based at least in part on the pressure measurement received from the pressure sensor,
wherein the pressure bag is adjacent to an intravenous fluid supply bag containing an intravenous fluid, and
wherein in an instance in which a fluid is pumped into the inlet port of the pressure bag, pressure is applied to the intravenous fluid supply bag, such that, intravenous fluid is ejected from an outlet port into the intravenous fluid supply tube; and
altering the output of the pump based at least in part on the pressure measurement received from the pressure sensor.

12. The method of claim 10, wherein the intravenous fluid supply tube is fluidly connected to a blood vessel of a patient and wherein the pressure sensor measures the pressure of the intravenous fluid in the intravenous fluid supply tube between the intravenous fluid supply bag and the blood vessel of the patient.

13. The method of claim 11, wherein the controller regulates the output of the pump such that the pressure of the intravenous fluid in the intravenous fluid supply tube is greater than a blood pressure of the patient.

14. The method of claim 10, wherein determining the output of the pump further comprises comparing the pressure measurement received from the pressure sensor to a minimum pressure threshold, and reconfiguring the output of the pump if the measurement received from the pressure sensor is below the minimum pressure threshold.

15. The method of claim 10, wherein altering the output of the pump comprises activating and deactivating the pump.

16. The method of claim 10, further comprising transmitting the plurality of pressure measurements of the pressure sensor for display on a display device.

17. A computer program product for regulating a pressure of an intravenous fluid supply, the computer program product comprising at least one non-transitory computer-readable storage medium having computer-readable program code portions stored therein, the computer-readable program code portions comprising an executable portion configured to:
receive a pressure measurement of a plurality of pressure measurements from a pressure sensor,
wherein the pressure sensor is positioned to measure the pressure within an intravenous fluid supply tube, and
wherein the intravenous fluid supply tube is fluidly connected to an outlet port of an intravenous fluid supply bag;
determine an output of a pump, fluidly connected to an inlet port of a pressure bag, based at least in part on the pressure measurement received from the pressure sensor,
wherein the pressure bag is adjacent to an intravenous fluid supply bag containing an intravenous fluid, and
wherein in an instance in which a fluid is pumped into the inlet port of the pressure bag, pressure is applied to the intravenous fluid supply bag, such that, intravenous fluid is ejected from an outlet port into the intravenous fluid supply tube; and
alter the output of the pump based at least in part on the pressure measurement received from the pressure sensor.

18. The computer program product of claim 17, wherein the intravenous fluid supply tube is fluidly connected to a blood vessel of a patient and wherein the pressure sensor measures the pressure of the intravenous fluid in the intravenous fluid supply tube between the intravenous fluid supply bag and the blood vessel of the patient.

19. The computer program product of claim 17, wherein determining the output of the pump further comprises comparing the pressure measurement received from the pressure sensor to a minimum pressure threshold, and reconfiguring the output of the pump if the measurement received from the pressure sensor is below the minimum pressure threshold.

20. The computer program product of claim 17, wherein altering the output of the pump comprises activating and deactivating the pump.
